(19) United States
(12) Patent Application Publication
(10) Pub. No.: US 2006/0259676 A1
(43) Pub. Date: Nov. 16, 2006

(54) Blood glucose meter/modem interface arrangement

(75) Inventors: John W. Oberding, Clearwater, FL (US); Glen E. Geiger, St. Petersburg, FL (US); Kenneth D. White, Cocoa, FL (US); Warren H. Miller, Palm Bay, FL (US); Reeder N. Ward, Melbourne, FL (US)

Correspondence Address: ALLEN, DYER, DOPPELT, MILBRATH & GILCHRIST P.A., 1401 CITRUS CENTER 255 SOUTH ORANGE AVENUE, P.O. BOX 3791, ORLANDO, FL, 32802-3791, US

(73) Assignee: NORTH AMERICAN TECHNOLOGY SERVICES, INC., Pinellas Park, FL (US)

(21) Appl. No.: 11/128,587

(22) Filed: May 13, 2005

(51) Int. Cl. G06F 13/00; A61B 5/00

(52) U.S. Cl. 710/303; 600/365

(57) ABSTRACT

An interface between a communication modem and a hand-held, battery-operated information collection device, such as a blood glucose meter comprises a support cradle into which blood glucose meter is placed. A mechanical translation unit within the cradle then automatically rotates a cam-driven, pivotable swing arm on which an electrical connector to modem memory is mounted. The swing arm follows a spiral groove in the cam, so as to fully insert the electrical connector into the data port receptacle of the meter. After data has been downloaded from the meter into modem memory, the mechanical translation unit causes reverse rotation of the cam, so as to retract the electrical connector out of the data port receptacle, whereupon the meter may be removed by the user.

### FIELD OF THE INVENTION

The present invention relates in general to information storage and retrieval systems and subsystems therefor, and is particularly directed to an interface between a communication modem and a hand-held, battery-operated information collection device, such as a blood glucose meter, that enables information representative of the results of data collection (blood glucose analysis) performed by and stored in the device to be automatically coupled to a communication modem for transmission to a processing site, such as may be installed at a doctor's office, clinic or other remote monitoring medical facility to which collected patient data is reported.

### BACKGROUND OF THE INVENTION

Diabetic patients typically monitor their blood glucose levels at home or at other non-clinical sites by means of disposable test strips and a battery-powered, hand-held analyzer (blood glucose meter). As a non-limiting example, such a meter may comprise a Home Diagnostics Blood Glucose Meter, Model No. SIQ452, which has a configuration depicted in the front perspective view of FIG. 1. As shown therein, the front 10 of the blood glucose meter 1 has a blood sample strip-receiving region 11, into which the user inserts a disposable test strip containing a blood sample for analysis by the meter. The front of the meter also has a display screen 12 that displays to the user information, such as operating instructions and the results of the blood glucose analysis.

A side region 13 of the meter contains a data communication port 14, which is adapted to receive a communication cable connector, through which the user may couple the meter to a data terminal device, such as a computer, or to a data communication device, such as a wireline or wireless modem. This dataport connection allows the results of a blood glucose analysis performed and stored by the meter to be reported via a telecommunication link to a healthcare facility that keeps track of the patient's progress over the course of a blood glucose treatment program.

A significant shortcoming associated with the use of devices of the type described above is the fact that the user, particularly an elderly person, may not have access to a computer or an associated modem, or be familiar with how to use such equipment. Indeed, very few patients use computer capture of data, and even fewer healthcare providers have direct access to this clinically valuable information. The only recourse is for the user to attempt to call a healthcare provider's information collection site with the information displayed on the screen—a chore that is problematic at best.

### SUMMARY OF THE INVENTION

In accordance with the present invention, the task of reporting the results of a blood glucose analysis, as performed by a blood glucose measurement meter of the type described above, to a remote monitoring facility, is considerably simplified, by means of a blood glucose meter/modem interface arrangement, which is configured to automatically download and transmit the results of the blood glucose analysis performed by the meter to a remote facility with minimum effort on the part of the user. All that is required of the user is to take a blood sample measurement with the blood glucose meter and then place the meter into a modem-containing, meter support and connection cradle.

To this end; the data collection meter/modem interface arrangement of the invention comprises a support cradle that is configured to receive and retain a (blood glucose) meter of the type described above. Once the meter has been placed in the cradle, a mechanical translation unit within the cradle automatically rotates a cam-driven, pivotable swing arm on which an electrical connector is mounted. The swing arm follows a spiral groove in the cam, that causes the electrical connector to be fully inserted into the data port receptacle of the meter. At the same time, an indicator on the cradle is illuminated to inform the user that the interface is in use.

The cradle also contains a data extraction unit, to which the electrical connector is coupled, and which is operative, in response to the electrical connector being inserted into the data port, to automatically download, into a modem memory, data that has been collected and stored by the meter. The modem may then send the data to a remote healthcare facility. After the data has been downloaded from the meter into the modem memory, the mechanical translation unit proceeds to cause reverse rotation of the cam, so as to translate the swing arm in a reverse direction, that is effective to remove or back the electrical connector out of the data port receptacle. The illumination of the indicator on the cradle is then extinguished to advise the user that the meter may be removed from the cradle.

### BRIEF DESCRIPTION OF THE DRAWINGS

FIG. 1 is a front perspective view of a conventional battery-powered blood glucose meter having a display screen and data communication port;

FIGS. 2, 3 and 4 are respective front, rear and side cutaway perspective views of the housing configuration of the data collection meter/modem interface arrangement of the invention;

FIGS. 5, 6, 7, 8 and 9 are respective top, end, side, perspective and exploded views of the data collection meter/modem interface arrangement of the invention;

FIGS. 10 and 11 are respective perspective and plan views of a gear-cam;

FIG. 12 shows a projected overlay of the swing arm, the gear-cam, the cam drive motor and blood glucose meter for respective fully removed and fully inserted positions of the electrical connector with respect to the dataport receptacle of the meter; and

FIG. 13 is a front perspective view of the invention, showing the meter as it is inserted into the cradle area of the support cradle.

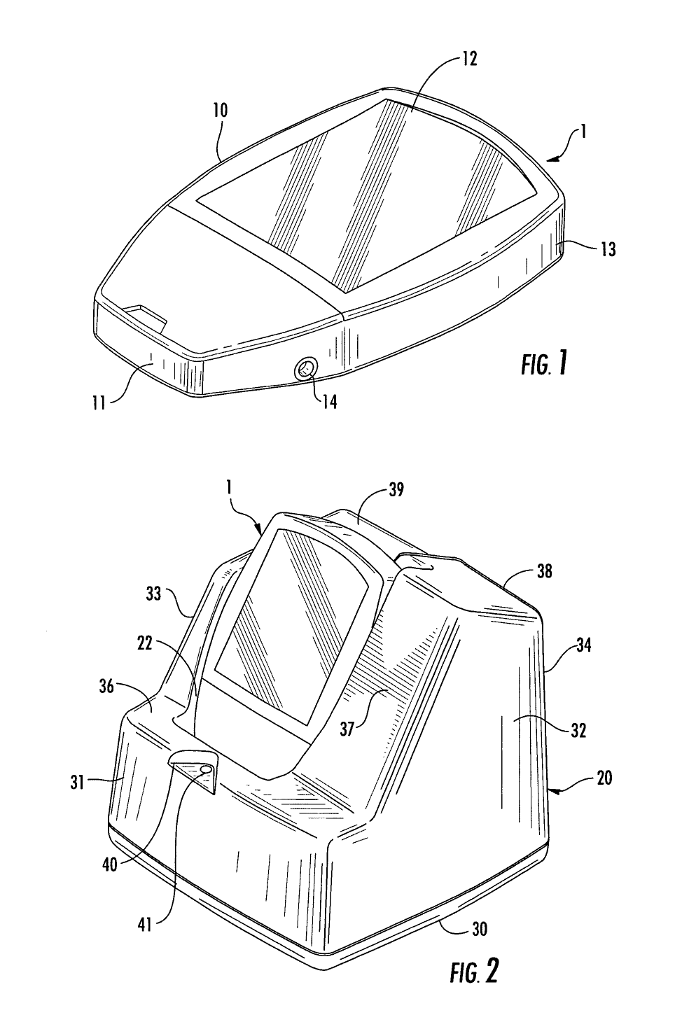

### DETAILED DESCRIPTION

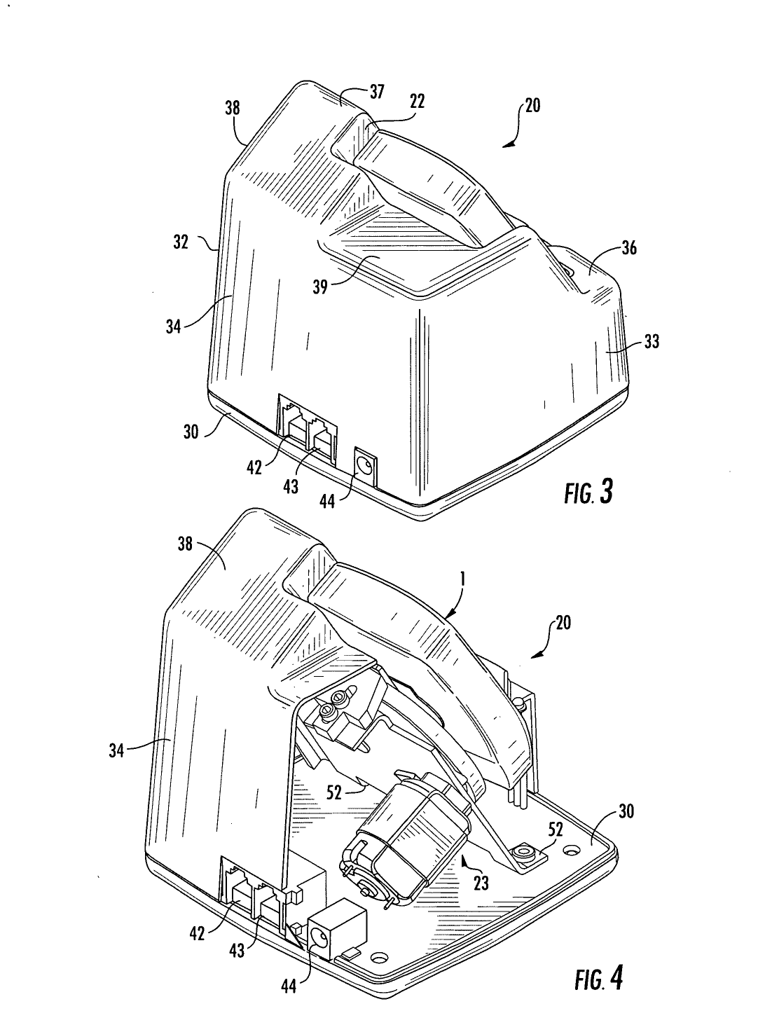

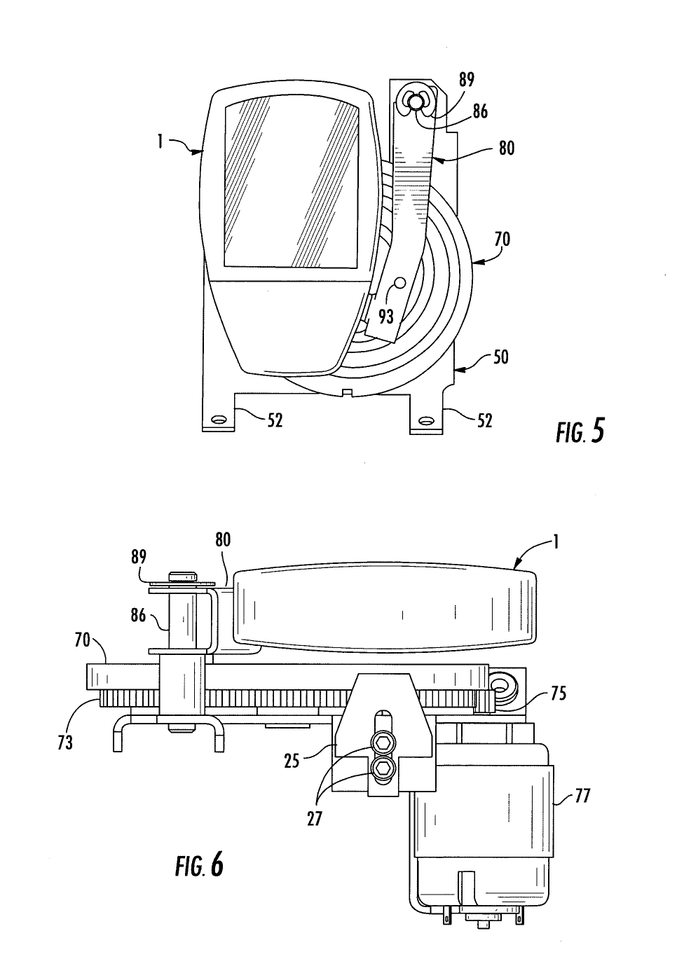

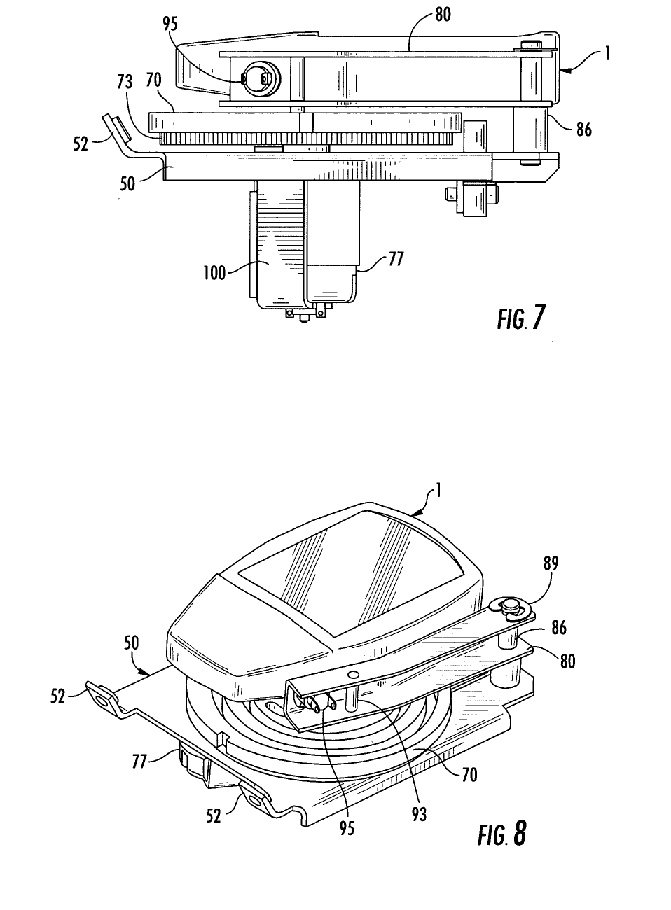

Attention is now directed to FIGS. 2, 3 and 4, which are respective front, rear and side cutaway perspective views of the housing configuration of the data collection meter/modem interface arrangement of the invention, as well as FIG. 13, which is a front perspective view, showing the manner of insertion of the meter into a meter cradle area of the support cradle. In particular, these Figures diagrammatically depict the general architecture of a support cradle 20, into an inclined meter insertion slot, or meter cradle area 22 of which a hand-held data collection device (e.g., blood glucose meter) 1 has been placed. As shown therein, support cradle 20 has a base 30, from which extend a lower front wall 31, an adjoining pair of spaced apart sidewalls 32 and 33, and a rear wall 34. The lower front wall 31 terminates at a shelf 36, which extends to an inclined upper front wall 37, into which the inclined meter insertion slot 22 is formed. As shown in the cutaway view of FIG. 4, and as will be detailed below with reference to FIGS. 5-12, inclined meter insertion slot 22 contains a mechanical translation unit 23, which is operative, in response to placement of a meter into the meter insertion slot, to automatically and controllably rotate a cam-driven, pivotable swing arm, on which an electrical connector is mounted, such that the electrical connector will be fully inserted into the data port receptacle of the meter.

The top of the support cradle includes an inclined top portion 38 that adjoins the sidewall 32, rear wall 34 and inclined upper front wall 37, and a generally horizontal top portion 39 that adjoins the sidewall 33, rear wall 34 and the inclined upper front wall 37. The distance between the top of the horizontal top portion 39 and the bottom of the meter insertion slot 22 at a stop thereof is less than the length of the meter, so as to provide sufficient space directly above the horizontal top portion 39 for the user to grasp the meter during its insertion into and removal from the cradle 20. A recess 40 is formed in a region adjoining the lower front wall 31 and the shelf 36, and contains an indicator 41, such as a light emitting diode (LED), which is used to inform the user when the interface is in use. The reverse side (rear wall 34) of the cradle contains a pair of telecommunication receptacles 42 and 43, and a DC power supply receptacle 44. The upper portion of the meter cradle area 22 has an optic sensor 25, that “sees” the meter 1 dropped into place in the meter cradle area 22 and, in response thereto, tells the on-board electronics to start a motor that rotates a cam that drives a swing arm to insert a jack into the meter's data connector 14.

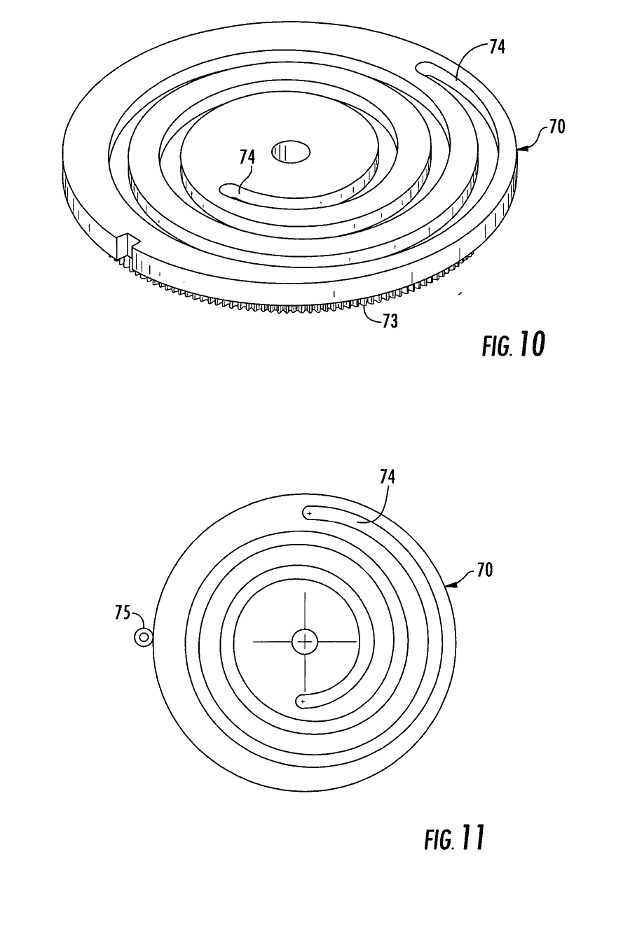

Details of the mechanical translation unit 23 are shown in FIGS. 5, 6, 7, 8 and 9, which are respective top, end, side, perspective and exploded views thereof, and FIG. 10, which is a perspective view of a gear-cam. FIGS. 5-8 also show the spatial relationship of the meter 1 relative to the components of the mechanical translation unit, when the meter has been inserted into the meter insertion slot of the cradle. In addition, these Figures show the position of the swing arm and the electrical connector affixed thereto for the fully inserted condition of the electrical connector relative to the dataport receptacle 14 of the meter.

Support for the various components of the mechanical translation unit is provided by way of a generally rectangular frame member 50, which is mounted to the base 30 of the interface by means of a pair of obtuse angled feet/tabs 52 and an angle brace 53, which is attached to the frame 50 by way of a screw 55. The feet/tabs 52 are affixed to the housing base 30 by way of fasteners that pass through apertures in the feet/tabs. The frame 50 includes a circular aperture 61, which is sized and located to receive a bearing 62 which supports a gear shaft 63, upon which a gear-cam 70 is mounted on a top side of the frame 50 for rotation about a gear-cam axis 72, coincident with the center of circular aperture 61. A C-clip 64 retains gear shaft 63 in bearing 62.

As shown in the perspective view of FIG. 10, gear-cam 70 has a plurality of gear teeth 73 around its circumferential edge, and contains a spiral groove 74 formed in the circular body thereof. Gear teeth 73 are engaged by a pinion gear 75 of the output shaft 76 of a drive motor 77, that is mounted to an underside of the frame 50, as will be described. The spiral groove 74 of the gear-cam 70 is sized to receive a cam follower pin 93 carried by a swing arm 80, and is used to guide the travel path the swing arm, as it rotates about a pivot axis 82. Pivot axis 82 is coincident with the center of a circular aperture 84 in frame 50, and is sized to receive a swing arm pivot shaft 86, upon which the swing arm 80 is mounted.

Swing arm 80 has a generally horizontal U-shaped cross-section and includes a first pair of coaxial pivot shaft mounting bores 81 and 83 in respective top and bottom sides 85 and 87 thereof. Pivot shaft mounting bores 81 and 83 are sized to fit upon and provide rotational support for the swing arm pivot shaft 86. A C-clip 89 is used to secure the swing arm 80 to the pivot shaft 86. The swing arm 80 further includes a second pair of coaxial bores 91 and 92 in the respective top and bottom sides 85 and 87 thereof. Bores 91 and 92 are sized to receive the cam follower pin 93, which rides in the spiral groove 74 of gear cam 70, as described above. A generally distal end 88 of an intermediate side 90 of the swing arm 80, which adjoins sides 85 and 87 thereof, contains an aperture 94, that is sized to receive an electrical connector 95 for controlled insertion into and removal from the dataport receptacle 14 of the meter 1. The electrical connector 95 is coupled via an interconnection cable to a modem installed within the support cradle 20 and connected to telecommunication receptacles 42 and 43.

As pointed out above, the gear teeth 73 of the gear-cam 70 are engaged by the pinion gear of an output shaft of drive motor 77 that is mounted to the underside of the frame 50 by means of a motor retention bracket 100. For this purpose, motor retention bracket 100 is secured to the underside of the frame 50 by way of a suitable fastener, such as a screw 101, and the like, which passes through a slot 103 in a top portion 105 of the bracket, and is screwed into threaded bore 108 of the frame proper. A drive motor 77 is supported between the top portion 105 and a bottom portion 111 of the motor bracket 100. Motor 110 has an output or drive shaft 76 that passes through an aperture 114 in the frame 50, and a pinion gear 75 is affixed to a distal end 116 of the motor's output shaft 76 for engagement with the gear teeth 73 of the gear-cam 70. An eccentric adjustment 115 provides for adjustment of the gear teeth 73 of cam 70 relative to the teeth of pinion gear 75. Optic sensor 25 is mounted to frame bracket 26 by screws 27.

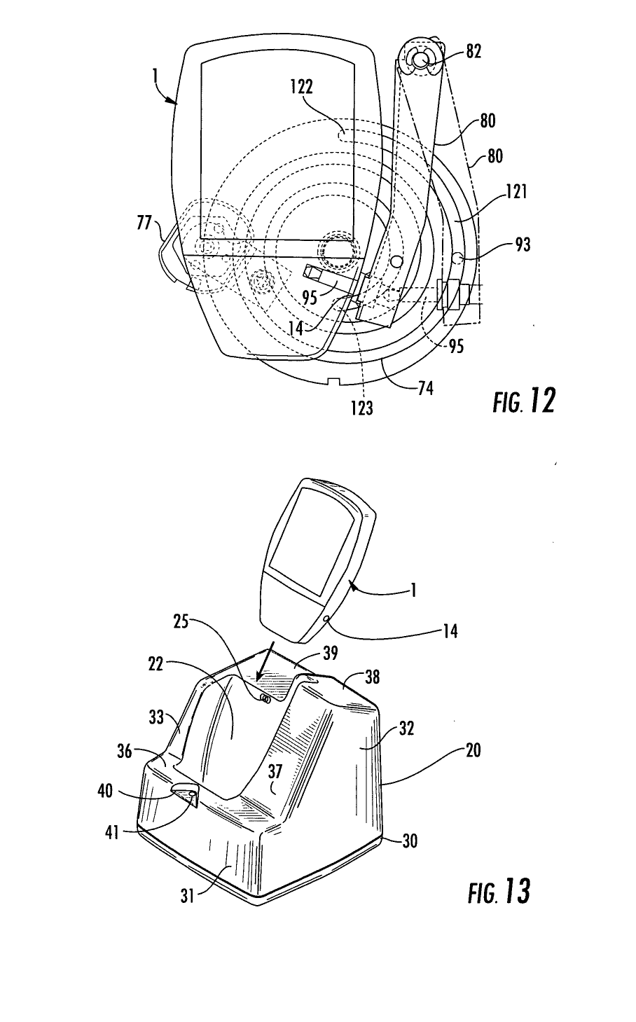

Operation of the meter/modem interface of the present invention may be readily understood by reference to FIG. 11, which is a diagrammatic plan view of the gear-cam 70 showing its spiral groove 74, and FIG. 12, which shows a projected overlay of the swing arm 80, the gear-cam 70, cam drive motor 77 and meter 1, for respective fully removed and fully inserted positions of the electrical connector 95 with respect to the dataport receptacle 14 of the meter 1. More particularly, the diagrammatic plan view of FIG. 11 shows the perimeter gear teeth of the gear-cam 70 being engaged by the pinion gear 75 at the distal end of the output drive shaft 76 of motor 77. FIG. 12 also shows the location of the cam follower pin 93 within an outer portion 121 of the spiral groove 74. This outer portion 121 terminates at an end termination 122, which corresponds to the fully retracted position of the swing arm 80, as shown in FIG. 12.

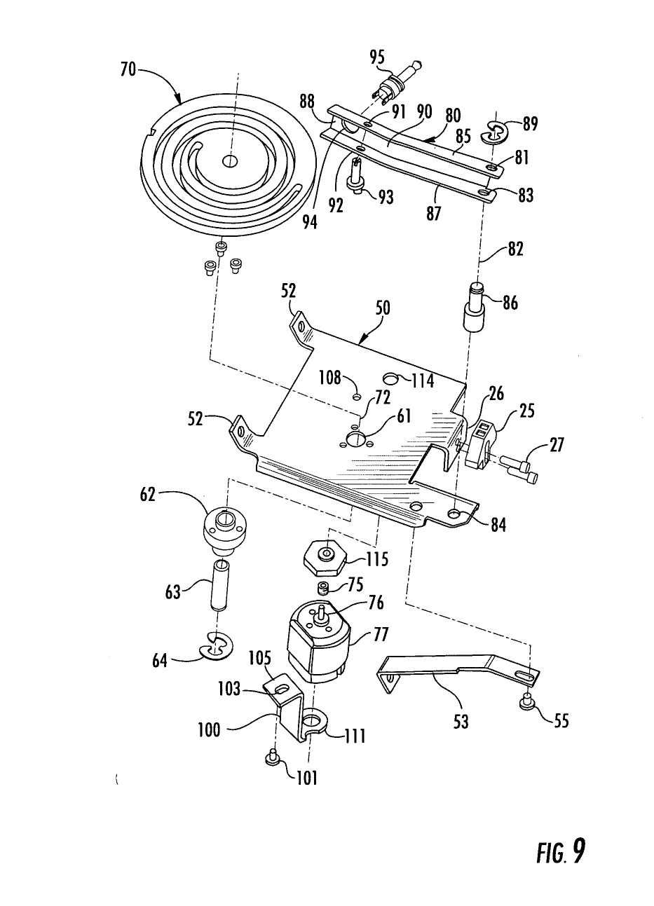

From this fully retracted position of the swing arm 80, driving the motor's output pinion gear 75 in a clockwise direction as viewed in FIGS. 11 and 12 will cause a counter-clockwise rotation of the gear-cam 70 about gear-cam axis 72. As the gear-cam 70 is rotated in a counter-clockwise direction, its spiral groove 74 is similarly rotated, so that the swing arm's cam follower pin 93 is caused to move along a travel path that translates the cam follower pin 93 closer to the center of the gear-cam. The radius of the travel path of the cam follower pin 93 is defined by the distance between the cam follower pin 93 and the pivot axis 82 of the swing arm 80. This travel path of cam follower pin 93 is such as to bring the electrical connector 95 into its fully inserted position into the dataport receptacle 14 of the meter 1, in response to the cam follower pin 93 reaching a second, inner termination 123 of the spiral groove 74. This fully inserted position of the electrical connector 95 into the dataport 14 of the meter 1 is shown additionally in FIGS. 5 and 8. As pointed out above, during this operation, the LED indicator 41 is illuminated to inform the user that the interface is in use.

In response to electrical connector 95 being pivoted into its fully inserted into the dataport receptacle 14, a data extraction unit within the cradle, and to which the electrical connector 95 is coupled, proceeds to download, into a modem memory, data that has been collected and stored by the meter. The modem may then send the data to a remote healthcare facility. After the data has been downloaded from the meter into the modem memory, the mechanical translation unit proceeds to cause reverse rotation of the cam 70, so as to translate the swing arm 80 in a reverse direction, that is effective to remove or back the electrical connector 95 out of data port receptacle 14.

More particularly, to remove the electrical connector 95 from its inserted position in the data port receptacle 14 to its removed position for the fully retracted position of the swing arm 80, the motor's output pinion gear 75 is driven in a counter-clockwise direction as viewed in FIGS. 11 and 12, which causes a clockwise rotation of the gear-cam 70 about gear-cam axis 72. As the gear-cam 70 is rotated in a clockwise direction, its spiral groove 74 is similarly rotated, so that the swing arm's cam follower pin 93 is caused to move along its travel path that translates the cam follower pin 93 farther away from the center of the gear-cam. This reverse travel path of the cam follower pin 93 is such as to bring the electrical connector 95 into its fully retracted position out of the dataport receptacle 14 of the meter 1, in response to the cam follower pin 93 reaching the first, outer termination 122 of the spiral groove 74. Illumination of the indicator 41 is then terminated, to advise the user that the meter-may be removed from the cradle.

As will be appreciated from the foregoing description, the task of reporting the results of a blood glucose analysis, as performed by a blood glucose measurement meter having a side located data port receptacle, to a remote monitoring facility, is considerably simplified in accordance with the blood glucose meter/modem interface arrangement of the invention, which is configured to automatically download and transmit the results of the blood glucose analysis performed by the meter to a remote facility with minimum effort on the part of the user. All that is required of the user is to take a blood sample measurement with the blood glucose meter and then place the meter into the modem-containing, meter support and connection cradle.

While we have shown and described an embodiment in accordance with the present invention, it is to be understood that the same is not limited thereto but is susceptible to numerous changes and modifications as known to a person skilled in the art. We therefore do not wish to be limited to the details shown and described herein, but intend to cover all such changes and modifications as are obvious to one of ordinary skill in the art.

What is claimed:

1. An apparatus for enabling information stored in a portable, battery-powered information collection device, having a data port receptacle that is adapted to receive an electrical connector through which data stored in said information collection device may be extracted, to be automatically extracted for transmission to a data reception site, comprising:
a support cradle that is configured to support said information collection device;
an electrical connector support device, that is configured to support said electrical connector;
a mechanical translation unit which is operative to cause relative motion between said information collection device as supported by said support cradle and said electrical connector support device, so as to cause said electrical connector to be mechanically and electrically coupled with said data port receptacle of said information collection device; and
a data extraction unit, coupled to said electrical connector and being operative, subsequent to said mechanical translation unit causing said electrical connector to be mechanically and electrically coupled with said data port receptacle of said information collection device, to automatically extract data stored in said information collection device for transmission to said data reception site.

2. The apparatus according to claim 1, wherein said portable, battery-powered information collection device comprises a blood glucose meter.

3. The apparatus according to claim 1, wherein said mechanical translation unit is operative to cause relative rotational motion between said information collection device as supported by said support cradle and said electrical connector support device, so as to cause said electrical connector to be mechanically and electrically coupled with said data port receptacle of said information collection device.

4. The apparatus according to claim 3, wherein said mechanical translation unit is operative to cause rotational motion of said electrical connector support device, so as to bring said electrical connector supported thereby into mechanical and electrical engagement with said data port receptacle of said information collection device.

5. The apparatus according to claim 4, wherein said mechanical translation unit comprises a pivotable swing arm, which is pivotable about an axis of rotation and to which said electrical connector is affixed, and a mechanical drive unit, coupled to said swing arm and being operative to pivot said swing arm about said axis and bring said electrical connector into mechanical and electrical engagement with said data port receptacle of said information collection device.

6. The apparatus according to claim 5, wherein said mechanical drive unit comprises a rotatable cam to which is pivotable swing arm is coupled, and a drive motor coupled to said rotatable cam and being operative to rotate said cam so as to cause said swing arm to pivot about said axis and bring said electrical connector into mechanical and electrical engagement with said data port receptacle of said information collection device.

7. The apparatus according to claim 6, wherein said rotatable cam contains a spiral groove to which a cam follower of said pivotable swing arm is coupled, and wherein said drive motor is coupled to a peripheral portion of said rotatable cam and being operative to rotate said cam so as to cause said cam follower to be displaced by said spiral groove and thereby cause said swing arm to pivot about said axis and bring said electrical connector into mechanical and electrical engagement with said data port receptacle of said information collection device.

8. The apparatus according to claim 1, wherein said electrical connector support device, said mechanical translation unit and said data extraction unit are supported by said support cradle.

9. The apparatus according to claim 1, wherein said mechanical translation unit is operative to cause rotational motion of said electrical connector support device in response to said information collection device being placed in said support cradle.

10. The apparatus according to claim 9, wherein said data extraction unit is operative to extract data stored in said information collection device, in response to said electrical connector being mechanically and electrically coupled with said data port receptacle of said information collection device.

11. The apparatus according to claim 10, wherein said data extraction unit includes memory into which said data extracted from said information collection device is stored.

12. The apparatus according to claim 11, further including a modem, coupled to said data extraction unit, said modem being operative to transmit data extracted from said information collection device and stored in said memory to a data collection site remote with respect to said apparatus.

13. The apparatus according to claim 11, wherein said mechanical translation unit is operative, in response to said data being extracted from said information collection device and stored in said memory of said data extraction unit, to cause relative reverse motion between said information collection device and said electrical connector support device, and thereby mechanically and electrically decouple said electrical connector from said data port receptacle of said information collection device.

14. An apparatus for automatically extracting information stored in a battery-powered portable information collection device, said information collection device having a data port receptacle that is adapted to receive an electrical connector through which data stored in said information collection device may be read out from said information collection device, comprising:
a support cradle that is configured to receive and support said portable information collection device;
an electrical connector support device, that is configured to support said electrical connector in a manner that provides for displacement of said electrical connector relative to said data port receptacle;
a mechanical displacement control unit which is operative, in response to placement of said information collection device in said support cradle, to controllably displace said electrical connector support device, so as to bring said electrical connector into mechanical and electrical engagement with said data port receptacle of said information collection device; and
a data extraction unit, coupled to said electrical connector and being operative, subsequent to said mechanical translation unit causing said electrical connector to be mechanically and electrically engaged coupled with said data port receptacle of said information collection device, to automatically download data stored in said information collection device into a communication device for transmission to a data reception site.

15. The apparatus according to claim 14, wherein said battery-powered, portable information collection device comprises a blood glucose meter.

16. The apparatus according to claim 14, wherein said mechanical translation unit is operative to allow bidirectional pivotal motion of said electrical connector support device, so as to controllably bring said electrical connector supported thereby into mechanical and electrical engagement with said data port receptacle of said information collection device in response to said information collection device being placed in said support cradle and, subsequent to reading out of data from said information collection device by said data extraction unit, to controllably retract said electrical connector supported thereby out of mechanical and electrical engagement with said data port receptacle of said information collection device.

17. The apparatus according to claim 16, wherein said mechanical translation unit comprises a pivotable swing arm, which is pivotable about an axis of rotation and to which said electrical connector is affixed, and a mechanical drive unit, coupled to said swing arm and being operative to controllably pivot said swing arm about said axis and controllably bring said electrical connector into and out of mechanical and electrical engagement with said data port receptacle of said information collection device.

18. The apparatus according to claim 17, wherein said pivotable swing arm is coupled to a rotatable cam containing a spiral groove to which a cam follower of said pivotable swing arm is coupled, and wherein a drive motor is coupled to a peripheral portion of said rotatable cam and is operative to controllably bidirectionally rotate said cam so as to cause said cam follower to be displaced by said spiral groove, and thereby cause said swing arm to pivot about said axis and bring said electrical connector into and out of mechanical and electrical engagement with said data port receptacle of said information collection device.

19. The apparatus according to claim 14, wherein said data extraction unit includes memory into which said data read out of said information collection device is stored for transmission to said data reception site.

20. The apparatus according to claim 19, further including a modem, coupled to said data extraction unit, said modem being operative to transmit data extracted from said information collection device and stored in said memory to said data collection site.

21. The apparatus according to claim 11, wherein said mechanical translation unit is operative, in response to data being read out of said information collection device and stored in said memory of said data extraction unit, to cause relative reverse pivotal displacement between said information collection device and said electrical connector support device, and thereby mechanically and electrically decouple said electrical connector from said data port receptacle of said information collection device.

22. A method for automatically extracting information stored in a battery-powered portable information collection device, said information collection device having a data port receptacle that is adapted to receive an electrical connector through which data stored in said information collection device may be read out from said information collection device, comprising the steps of:
(a) inserting said portable information collection device into a support cradle therefor;
(b) in response to placement of said portable information collection device into said support cradle in step (a), automatically rotating an electrical connector support device for said electrical connector, so as to bring said electrical connector into electrical and mechanical engagement with said data port receptacle of said information collection device; and
(c) in response to said electrical connector being brought into electrical and mechanical engagement with said data port receptacle of said information collection device in step (c), automatically downloading data stored in said information collection device to a communication device for transmission to a data reception site.

23. The method according to claim 22, wherein said portable, battery-powered information collection device comprises a blood glucose meter.

24. The method according to claim 22, further including the step of:
(d) in response to downloading of data from said information collection device in step (c), automatically rotating said electrical connector out of mechanical and electrical engagement with said data port receptacle of said information collection device.

25. The method according to claim 24, wherein said electrical connector is supported by a cam-driven, pivotable swing arm, and wherein step (b) comprises, in response to placement of said portable information collection device into said support cradle in step (a), automatically rotating said cam-driven, pivotable swing arm, so as to bring said electrical connector into electrical and mechanical engagement with said data port receptacle of said information collection device.

26. The method according to claim 25, wherein step (d) comprises, in response to downloading of data from said information collection device in step (c), automatically rotating said cam-driven, pivotable swing arm in a reverse direction, so as to retract said electrical connector out of electrical and mechanical engagement with said data port receptacle of said information collection device.
